(19) United States
(12) Patent Application Publication
(10) Pub. No.: US 2006/0155307 A1
(43) Pub. Date: Jul. 13, 2006

(54) Method of feeding a suture element

(76) Inventor: Theodor Gerhard Rosch, Pretoria (ZA)

Correspondence Address: FITCH EVEN TABIN AND FLANNERY, 120 SOUTH LA SALLE STREET, SUITE 1600, CHICAGO, IL, 60603-3406, US

(21) Appl. No.: 10/531,513

(22) PCT Filed: Oct. 10, 2003
(86) PCT No.: PCT/IB03/04489

§ 371 Date: Nov. 1, 2005

(30) Foreign Application Priority Data
Oct. 17, 2002 (ZA) 2002/8423

(51) Int. Cl. A61B 17/10

(52) U.S. Cl. 606/139

(57) ABSTRACT

This invention relates to a method of feeding a suture element (24), which method includes using fluid pressure to displace the suture element (24). The invention extends to a suture element feeding device (10) which includes a body (12) defining a fluid flow path (20) and a fluid inlet (502) whereby a fluid under pressure can be fed into the fluid flow path (20). The body (12) further defines a suture element inlet whereby a suture element (24) can be fed into the fluid flow path (20) for displacement along the fluid flow path (20) together with the fluid, and an outlet (18) whereby the fluid and suture element (24) can be fed from the device (10), the fluid flow path (20) connecting the fluid inlet (502), the suture element inlet and the outlet (18) in flow communication. The invention relates also to a medical implement.

THIS INVENTION relates to a method of feeding a suture element. It further relates to a suture element feeding device and to a medical implement.

According to one aspect of the invention, there is provided a method of feeding a suture element, which method includes using fluid pressure to displace the suture element.

The method may include entraining the suture element in a fluid flow stream to displace the suture element in the direction of fluid flow.

By suture element is to be understood a length of flexible material suitable for use in suturing, i.e. in forming stitches, or the like. The element may be of so-called ‘soft’ suture material, i.e. typically of a braided, multi-filament type.

More particularly, the method may include

disposing the suture element in a fluid flow path; and

causing fluid to flow under pressure along the flow path thereby to displace the suture element along the flow path.

In one embodiment of the invention, disposing the suture element in the fluid flow path may include feeding it into an inlet end of the fluid flow path together with the fluid.

In another embodiment of the invention, disposing the suture element in the fluid flow path may include feeding the suture element through a suture element feed path which intersects the fluid flow path at a position intermediate an inlet end and an outlet end thereof.

The method may include inhibiting flow of fluid through the suture element feed path in a direction opposite to the direction in which the suture element is to be fed.

The method may include forming at least one eye in the suture element along its length, the eye being configured to receive another suture element therethrough.

According to still another aspect of the invention, there is provided a suture element feeding device which includes

a body defining a fluid flow path and a fluid inlet whereby a fluid under pressure can be fed into the fluid flow path, a suture element inlet whereby a suture element can be fed into the fluid flow path for displacement along the fluid flow path together with the fluid, and an outlet whereby the fluid and suture element can be fed from the device, the fluid flow path connecting the fluid inlet, the suture element inlet and the outlet in flow communication.

The device may include fluid displacement means for displacing fluid along the fluid flow path. The device may include actuating means for selectively actuating displacement of the displacement means towards a displaced position in which fluid is displaced along the flow path by the displacement means.

The body may define a reservoir, for holding the fluid to be fed into the fluid flow path, the reservoir being connected in flow communication with the fluid inlet.

According to yet another aspect of the invention, there is provided a medical implement which includes a body defining

tissue penetration means for penetrating a tissue to be sutured;

a fluid flow path;

a fluid inlet whereby a fluid under pressure can be fed into the fluid flow path;

a suture element inlet whereby a suture element can be fed into the fluid flow path for displacement along the fluid flow path together with the fluid; and

an outlet whereby the fluid and suture element can be fed from the body,

the fluid flow path connecting the fluid inlet, the suture element inlet and the outlet in flow communication, and the outlet opening out of the body at a position proximate the tissue penetration means.

In one embodiment of the medical implement, the fluid inlet and the suture element inlet are provided by a single inlet into the fluid flow path. In this embodiment, the body may define a reservoir, for holding the fluid to be fed into the fluid flow path, the reservoir being connected or connectable in flow communication with the fluid inlet.

In another embodiment of the medical implement, the suture element inlet may open into the fluid flow path at a position intermediate the fluid inlet and the outlet. In this embodiment of the invention, the body may define a suture element feed path which intersects the fluid flow path at a position intermediate the fluid inlet and the outlet, the suture element feed path opening into the fluid flow path via the suture element inlet. In this embodiment, the medical implement may include releasable securing means for selectively releasing and securing a suture element received in the suture element feed path, for feed into the fluid flow path, thereby to permit control of the amount of suture element fed into the fluid flow path.

The implement may include tissue drive means for driving the tissue onto the tissue penetrating means.

The invention will now be described, by way of example, with reference to the accompanying diagrammatic drawings.

In the drawings,

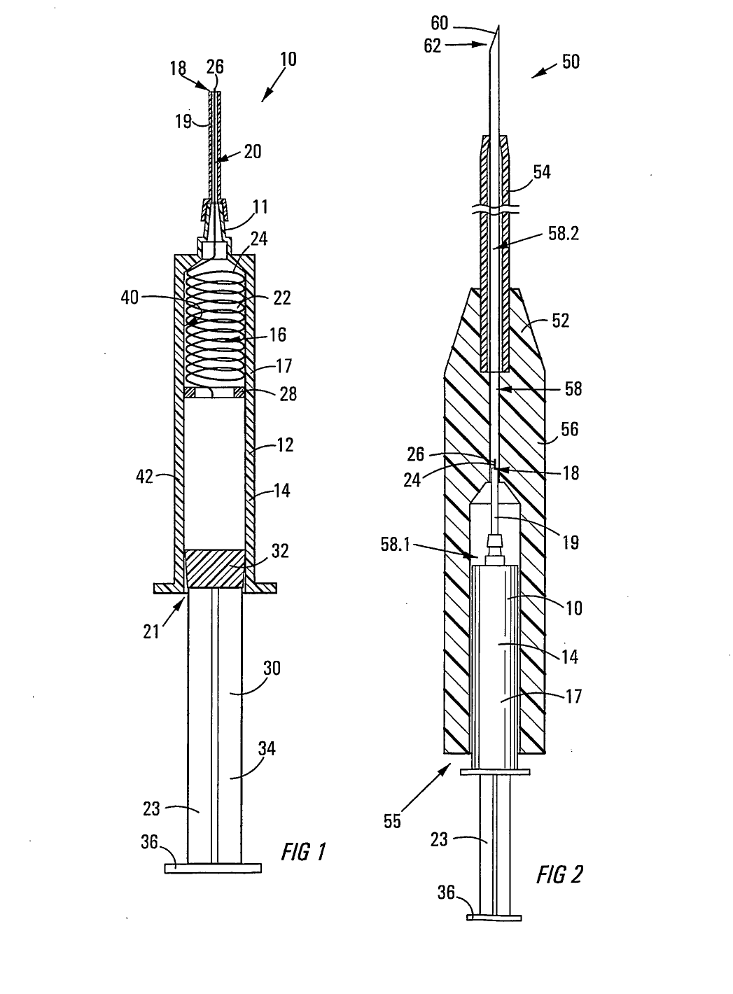

FIG. 1 shows a longitudinal sectional view of a suture element feeding device in accordance with the invention;

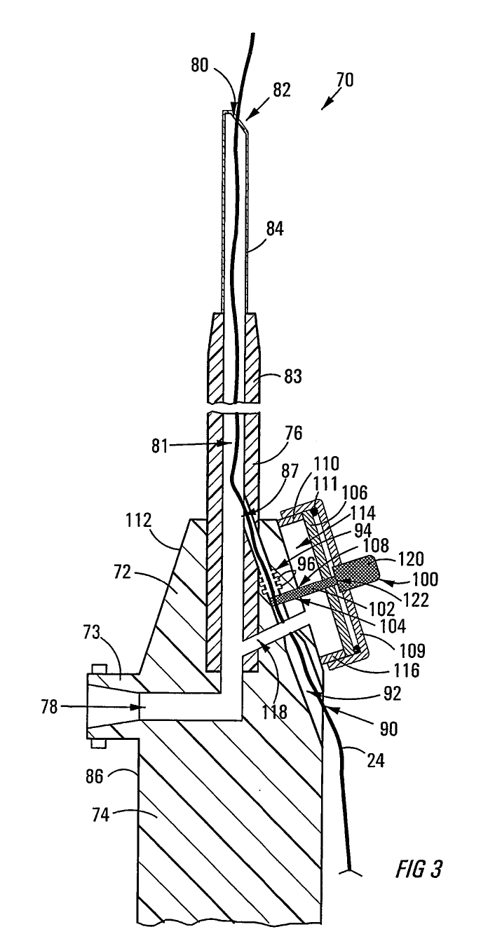

FIG. 2 shows a longitudinal sectional view of a medical implement in accordance with the invention;

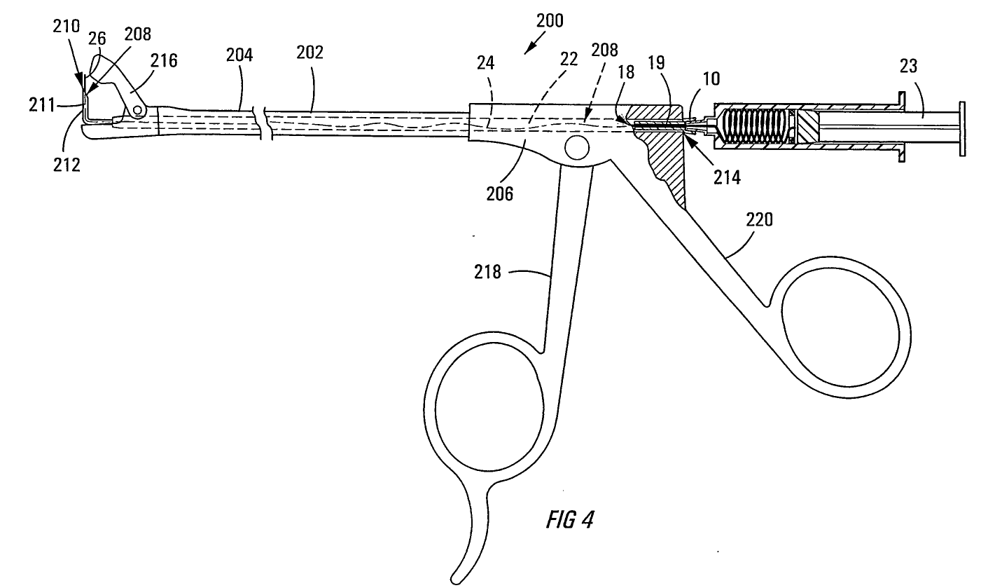

FIG. 3 shows a longitudinal sectional view of another medical implement in accordance with the invention;

FIG. 4 shows a part-sectioned side view of still another medical implement in accordance with the invention;

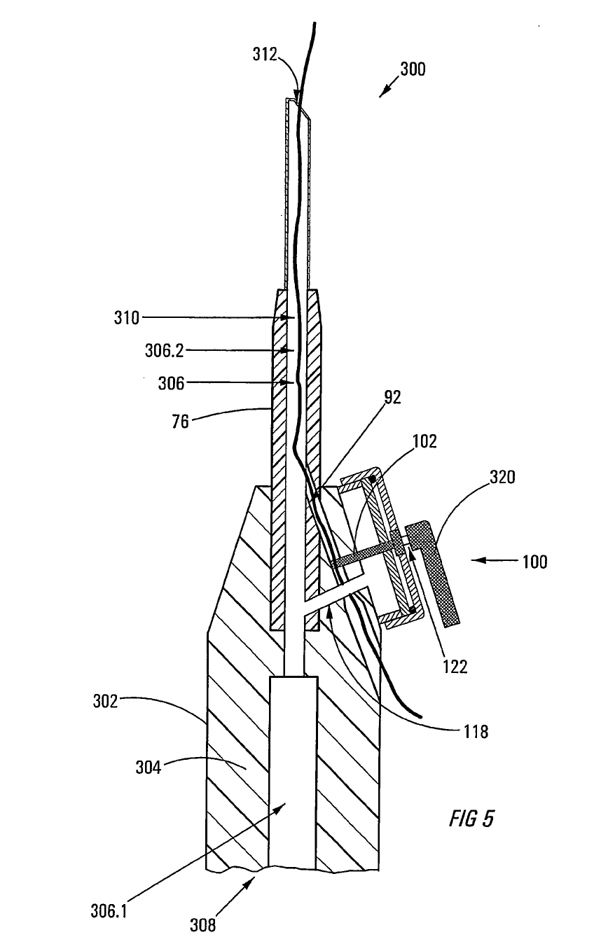

FIG. 5 shows a longitudinal sectional view of yet another medical implement in accordance with the invention;

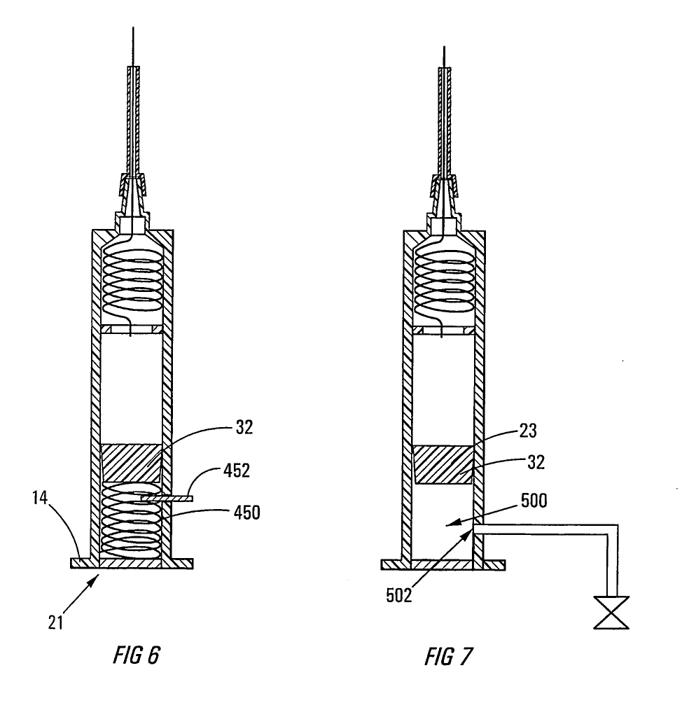

FIG. 6 shows a longitudinal sectional view part of another embodiment of the feeding device of FIG. 1; and

FIG. 7 shows a longitudinal sectional view of part of still another embodiment of the feeding device of FIG. 1.

In FIG. 1 of the drawings, reference numeral 10 refers generally to a suture element feeding device in accordance with the invention. The feeding device 10 includes a body 12 provided by a syringe 14.

The syringe 14 includes a circular cylindrical hollow barrel 17, defining a reservoir 16. A nipple 11 protrudes from one end of the barrel 17. An elongate needle 19 is mounted to the nipple 11, the free end of the needle 19 defining an outlet 18. A plunger 23 is snugly receivable in an open end 21 of the barrel 17 remote from the needle 19.

The plunger 23 includes a plunger body 30, having a central elongate stem 34 and a transverse radially outwardly extending disc 36 at one end thereof, providing a thumb plate by which a thumb of a user can depress the plunger 23. A piston 32 of a resiliently deformable material, typically silicone, is fitted over an end of the plunger body's stem 34 opposed to the disc 36. The piston 32 is configured to be sealingly and slidably receivable in the barrel 17.

In certain preferred embodiments of the invention, the feeding device 10 includes actuating means for selectively actuating displacement of the plunger 23 towards a displaced position, i.e. a position in which the plunger 23 is fully inserted into the barrel 17. In one embodiment (FIG. 6), displacement of the plunger 23, comprising the piston 32, towards its displaced position is actuated by means of a spring 450 mounted in compression between the piston 32 and the end 21 of the barrel 17 and incorporating a release mechanism (eg. a pin 452 removably received through an opening defined in a wall of the barrel 17 and intercepting the spring 450) by which the spring 450 can be released selectively to actuate displacement of the plunger 23. In another embodiment, (FIG. 7) a cavity 500 is defined between the plunger 23 and the end 21, the end 21 of the barrel 17 being closed. The barrel 17 further defines an inlet 502 in flow communication with the cavity 500 and includes connecting means for connecting the inlet 502, and thus the cavity 500 in flow communication with a supply of pressurised fluid, eg. compressed air supply means (not shown). Displacement of the plunger 23 is then actuated by means of compressed air fed into the cavity.

The nipple 11 and needle 19 define a fluid flow path 20 connecting the reservoir 16 and outlet 18 in flow communication.

The reservoir 16 contains a fluid 22, typically a biocompatible (or physiological) fluid such as, for example, water, a saline solution, or the like. A suture element 24, typically a multi-filament suture element 24 in the form of a soft, braided thread and formed from a plurality of intertwined filaments, is arranged in coiled form, eg. in the form of a spiral, in the reservoir 16. A free end 26 of the suture element 24, proximate the needle 19, is positioned in the fluid flow path 20 defined by the nipple 11 and the hollow of the needle 19.

An annular stopper 28 is snugly received in the reservoir 16 to retain the pre-loaded suture element 24 in position and in its coiled form. The stopper 28 is provided by a ring of synthetic rubber or resin, such as, for example silicone, and is mounted in the barrel 17 to be slidably displaceable to a downstream position, ie. towards the nipple 11, as fluid 22 is discharged from the reservoir 16 through the fluid flow path 20 as the plunger 23 is displaced in a downstream direction towards the nipple 11.

An internal surface 40 of the barrel 17 is shaped to facilitate feeding of the suture element 24 into and along the flow path 20 defined by the needle 19. To this end, the internal surface 40 tapers inwardly from the circular cylindrical side wall 42 towards an inlet of the nipple 11.

The needle 19 is typically blunt at its free end defining the outlet 18.

Reference is now made to FIG. 2 of the drawings, in which, unless otherwise indicated, the same reference numerals used above are used to designate similar parts. In FIG. 2, reference numeral 50 refers generally to a medical implement for piercing tissue. The implement 50 includes a body 52 comprising a needle portion 54 and a handle portion 56. A bore 58 extends through the body 52 opening out of opposed ends of the handle portion 56 and the needle portion 54.

The bore 58 has a portion 58.1, which extends longitudinally inwardly from a free end 55 of the handle portion 56 for part of its length, and a portion 58.2 of reduced diameter, which extends for the remainder of the body 52 and opens out of a free end 62 of the needle portion 54. The portion 58.1 is dimensioned such that at least part of the barrel 17 of a suture element feeding device 10 is receivable therein. The portion 58.2 is dimensioned so as to receive the needle 19 of the syringe 14 snugly therein when the barrel 17 is positioned in the portion 58.1.

It will be appreciated that a tip 60 of the needle portion 54, defined at the free end 62 of the needle portion 54, is typically sharp, to facilitate penetration of tissue to be sutured, and pointed. In the embodiment shown, the tip 60 is straight. Instead, the tip 60 may be curved at any radius, or of corkscrew form. Naturally, however, the tip 60 of the needle portion 54 may be of any suitable shape.

In use, typically during endoscopic surgery, and, more particularly, arthroscopic surgery, the tissue to be sutured (eg. muscle, ligament, tendon, or the like) is pierced by the tip 60 of the implement 50. The disc 36 of the syringe plunger 23 is depressed by a user of the implement 50 and the physiological fluid 22 is displaced under pressure from the reservoir 16 along the flow path 20 towards, and ultimately through, the outlet 18 of the syringe 14. The end 26 of the suture element 24 is displaced along with the fluid 22, entrained in the fluid 22, out of the outlet 18. As the free end 26 of the suture element 24 is displaced, it draws with it the remainder of the suture element 24, causing the suture element 24 to progressively unwind from its coiled form.

The fluid 22 is displaced under pressure, once it passes through the outlet 18, along the portion 58.2 of the bore 58 into the needle 54 and towards the tip 60 of the implement 50. The suture element 24, entrained in the fluid 22, is displaced with the fluid 22, partly by friction and partly by fluid pressure, until the free end 26 reaches the tip 60 where it exits the bore 58. In this way, the soft suture element 24 is passed through the tissue. The free end 26 of the suture element 24 may then be retrieved by use of suitable apparatus and a knot tied in the suture element 24 thereby to provide a suture/stitch.

It will be appreciated that, if a so-called “shuttle” is required by a user of the implement 50, i.e. the soft suture element 24 is to be used to pull another suture element along with it as it is displaced through the tissue, an eye may be formed at a position along the length of the first suture element 24 by piercing the multi-filamented braided suture element 24. The second suture element may then be received through the eye, such that the first suture element 24 provides the shuttle.

The implement 50 may be adapted to form part of a so-called suture punch, (see reference to FIG. 4 below) i.e. an implement having two jaws. A first fixed jaw will then incorporate the needle portion 54, a second jaw, displaceable relative to the first jaw, being configured to push the tissue to be repaired over the first jaw, i.e. over the needle tip 60.

Reference is now made to FIG. 3 of the drawings, in which reference numeral 70 refers generally to another medical implement providing a suture element feeding device in accordance with the invention, and, unless otherwise indicated, the same reference numerals used above are used to designate similar parts.

The implement 70 includes a body 72 having a roughly circular cylindrical handle portion 74 which tapers, defining a needle hub, towards a roughly circular cylindrical needle portion 76 of reduced diameter. The body 72 defines a fluid inlet 78 in the handle portion 74, a free end 82 of the needle portion 76 defining an outlet 80. The needle portion 76 is of stepwise reduced diameter, a shaft part 83 of the needle portion 76, proximate the handle portion 74, being of a greater diameter than a tip part 84, remote from the handle portion 74. The thicker shaft 83 serves to impart strength to the needle portion 76.

In the embodiment shown, the fluid inlet 78 is defined in the circular cylindrical side wall 86 of the handle portion 74. Naturally, the fluid inlet 78 may be defined elsewhere on the body 72 (see, for example, FIG. 5). A generally L-shaped fluid flow path 81 connects the fluid inlet 78 and the outlet 80 in flow communication, the fluid inlet 78 providing an inlet whereby a fluid under pressure can be injected into the fluid flow path 81.

The inlet 78 is connectable in flow communication with physiological fluid supply means (not shown). To this end, the body 72 includes connecting means 73, in the form of a Luer lock, defined on the body adjacent to the inlet 78 for connecting the fluid supply means, typically provided by a syringe, in flow communication with the inlet 78.

The body 72 further defines a suture element inlet 87 into the fluid flow path 81 at a position intermediate the fluid inlet 78 and the outlet 80. A suture element feed opening 90 is defined in a surface of the handle portion 74 at a position approximately diametrically opposed to the fluid inlet 78. A suture element feed path 92 extends from the opening 90 into the fluid flow path 81 opening into the fluid flow path 81 via the suture element inlet 87 and intersecting the flow path 81 at an acute angle at a position within the needle portion 76. In one embodiment (as shown in FIG. 3) baffle means 94, provided by a plurality of closely spaced areas 96 of increased diameter, is defined in the feed path 92 to inhibit a backflow of physiological fluid, supplied to the fluid flow path 81 via the fluid inlet 78, through the inlet 87 and along the feed path 92.

In use, a suture element 24 is inserted into the feed path 92 via the opening 90 and is manipulated along the feed path 92 through the suture element inlet 87 to be disposed within the flow path 81. Fluid 22 under pressure is injected into the flow path 81 via the fluid inlet 78 and is displaced along the flow path 81 toward the outlet 80, carrying with it the suture element 24, which is displaced partly by friction and partly by the fluid pressure

The implement 70 includes releasable securing means, generally indicated by reference numeral 100, for releasably securing the suture element 24 in position in the suture element feed path 92.

The securing means 100 includes a securing pin/rod 102 which is slidably displaceable within a bore 104, defined in the handle portion 74 at right angles to the feed path 92 and which intersects the feed path 92, selectively to intercept or be removed from the feed path 92. It will be appreciated that, when intercepting the feed path 92, the pin 102 abuts the suture element 24 thereby inhibiting displacement of the suture element 24 within the feed path 92. The pin 102 is resiliently biassed towards a rest position in which it intercepts the feed path 92 by resilient bias means provided by a diaphragm 106, which is pulled taut over a bore opening 108, defined in the handle portion 74, which leads into the bore 104. The diaphragm 106 is secured to the handle portion 74 by means of a circular cap 109 received over the diaphragm 106 and secured to the handle portion 74 by engagement with an annular ridge 110 defined on a tapering region of the outer surface 112 of the handle portion 74. An o-ring 111, of a diameter roughly corresponding to that of the annular ridge 110, is located between the diaphragm 106 and the cap 109 to retain the diaphragm 106 in position whilst maintaining a clearance space between the diaphragm 106 and the cap 109. The annular ridge 110 defines a central cavity 114 within its sidewall 116. A vent duct 118 diverges from the fluid flow path 81 and extends into the central cavity 114 of the annular ridge 110, intersecting the feed path 92, so that the central cavity provides a chamber into which part of the fluid 22 injected into the fluid inlet 78 flows, via the vent duct 118, upon injection into the fluid inlet 78. The chamber is at a pressure corresponding to the pressure of fluid in the flow path 81. The diaphragm 106 is displaceable outwardly under pressure of the fluid thereby to displace the pin 102 and withdraw it, at least partly, from the suture element feed path 92. The suture element 24 is, in this way, released for feed along the feed path 92 and flow path 81, respectively, under influence of the pressure of the fluid 22 injected into the flow path 81.

The securing means 100 further includes a knurled nut 120 which is fastened to an end 122 of the pin 102 remote from the feed path 92 which end 122 passes centrally through the diaphragm 106. The nut 120 is received through a central opening defined in the cap 109 and provides a finger hold by which the pin 102 can be manually withdrawn from the suture element feed path 92.

In use, the free end 82 of the needle portion 76 is passed through tissue to be repaired, ie. the tissue is penetrated by a user of the implement 70. A syringe containing a physiological fluid (not shown) is connected to the implement 70 at the fluid inlet 78 by means of the Luer lock 73 and a user of the implement 70 depresses a plunger of the syringe to eject its fluid contents into the flow path 81 via the inlet 78. The fluid 22 flows along the flow path 81, passing the point where the suture element feed path, and a suture element 24 fed along the feed path, intercept the flow path 81. Part of the injected fluid 22 is diverted along the vent duct 118 into the reservoir 114 defined by the annular ridge 110, where the fluid pressure build-up results in the displacement of the diaphragm 106 and pin 102 laterally outwardly of the feed path 92. The suture element 24 is thereby freed for displacement, under influence of the fluid 22 flowing along the flow path 81, along the flow path 81 and towards and out of the outlet 80 at the tip 84 of the implement. The suture element 24 is in this way passed through the tissue to be repaired. The suture can then be secured in position, eg. by tying a knot or in any suitable fashion.

Reference is now made to FIG. 4 of the drawings, in which reference numeral 200 refers generally to another medical implement in accordance with the invention, and, unless otherwise indicated, the same reference numerals used above are used to designate similar parts. The implement 200 includes a body 202 comprising a needle portion 204 and a handle portion 206. A bore 208 extends through the body 202, opening out of opposed ends 210,214 of the needle portion 204 and handle portion 206, respectively. At its free end 210, the needle portion 204 defines a sharp needle tip 212 which is generally L-shaped so that an end portion 211 of the needle tip 212 projects roughly at a right angle relative to the remainder of the needle portion 204. The bore 208 opens out of the end 210 of the needle portion 204 through the tip 212. The bore 208 is dimensioned, where it opens out of the opposed end 214 of the body 202 (i.e. out of the handle portion 206), so that the needle 19 of a suture element feeding device 10 is snugly receivable therein.

The needle portion 204 includes a jaw element 216 which is mounted at the end 210 of the needle portion 204 closely spaced from the needle tip 212 to be selectively laterally outwardly and inwardly displaceable relative to the needle tip 212 to grasp and push tissue over the needle tip 212.

The handle portion 206 includes a pair of relatively pivotally displaceable scissor-like handles 218, 220, the handle 218 being selectively displaceable towards or away from the handle 220 selectively to displace the jaw element 216 laterally outwardly or inwardly relative to the needle tip 212.

It will be appreciated that, in use, the handle 208 is displaced towards the handle 220 to displace the jaw element 216, having grasped tissue, towards and over the needle tip 212. The handle 218 may then be returned to a rest position thereby to displace the jaw element 216 laterally outwardly of the needle tip 212. A user of the implement 200 then depresses the syringe plunger 23 to displace a suture element 24, along with the physiological fluid 22, out of the outlet 18 of the syringe 14 and along the bore 208 towards the tip 212 of the implement 200, partly by friction and partly by fluid pressure, until the free end 26 of the suture element 24 reaches the tip 212 where it exits the bore 208 and is passed through the tissue.

In FIG. 5 of the drawings, reference numeral 300 refers generally to still another medical implement in accordance with the invention. The implement 300 is similar to the implement 70 (FIG. 3) and, unless otherwise indicated, the same reference numerals used above in relation to FIG. 3 designate similar parts.

The implement 300 includes a body 302, similar to the body 72 and having a handle portion 304 and a needle portion 76. A bore 306 extends through the body 302 opening out of opposed ends of the handle portion 304 and needle portion 76. A portion 306.1 of the bore 306 which extends longitudinally inwardly of a free end 308 of the handle portion 304 for part of its length, is dimensioned to receive a barrel of a syringe containing a physiological fluid (not shown) therein. A portion 306.2 of the bore 306 which extends through the remainder of the body 302 provides a fluid flow path 310 opening out of a free end 312 of the needle portion 76. It will be appreciated that the portion 306.1 provides an inlet via which physiological fluid ejected from the syringe can enter the flow path 310. The fluid inlet and the outlet 80 are thus connected in flow communication by a substantially linear flow path 310.

The suture element feed path 92 intercepts the fluid flow path 81 in the portion 306.2 of the bore 306. The releasable securing means 100 includes a lever 320 which is fastened to the end 122 of the pin 102 and is manually displaceable by a user between an engaged position, towards which it is resiliently biassed and in which the pin 102 engages a suture element 24 received in the suture element feed path 92 to secure the suture element 24 in position, and a released position, in which the pin 102 is withdrawn from the suture element feed path 92.

The Inventor believes that the devices/implements 10, 50, 70, 200, 300 in accordance with the invention will provide an effective means of feeding so-called soft suture material, of the braided multi-filament type, for suturing purposes. This in turn will dispense with the need for the use of more rigid monofilament suture elements with their associated more serious trauma effect on the tissue to be repaired.

1-17. (canceled)

18. A method of feeding a suture element, which method includes disposing the suture element in a fluid flow path by feeding the suture element through a suture element feed path, which intersects the fluid flow path at a position intermediate an inlet end and an outlet end thereof; and
causing fluid to flow under pressure along the flow path thereby to displace the suture element along the flow path in the direction of fluid flow.

19. The method as claimed in claim 18, which includes inhibiting flow of fluid through the suture element feed path in a direction opposite to the direction in which the suture element is to be fed.

20. The method as claimed in claim 18, which includes forming at least one eye in the suture element along its length, the eye being configured to receive another suture element therethrough.

21. The method as claimed in claim 18, which includes selectively releasing and securing the suture element in the suture element feed path thereby to control the amount of suture element fed into the fluid flow path.

22. A medical implement, which includes a body defining
tissue penetration means for penetrating a tissue to be sutured;
a fluid flow path;
a fluid inlet whereby a fluid under pressure can be fed into the fluid flow path;
an outlet whereby the fluid and suture element can be fed from the body, and
a suture element inlet which opens into the fluid flow path at a position intermediate the fluid inlet and the outlet, and whereby a suture element can be fed into the fluid flow path for displacement along the fluid flow path together with the fluid,
the fluid flow path connecting the fluid inlet, the suture element inlet and the outlet in flow communication, and the outlet opening out of the body at a position proximate the tissue penetration means.

23. The implement as claimed in claim 22, in which the body defines a reservoir, for holding the fluid to be fed into the fluid flow path, the reservoir being connected or connectable in flow communication with the fluid inlet.

24. The implement as claimed in claim 22, in which the body defines a suture element feed path which intersects the fluid flow path at a position intermediate the fluid inlet and the outlet, the suture element feed path opening into the fluid flow path via the suture element inlet.

25. The implement as claimed in claim 24, in which the implement includes releasable securing means for selectively releasing and securing a suture element received in the suture element feed path, for feed into the fluid flow path, thereby to permit control of the amount of suture element fed into the fluid flow path.

26. The implement as claimed in claim 22, which includes tissue drive means for driving the tissue onto the tissue penetrating means.

27. A medical implement, which includes a body defining tissue penetration means for penetrating a tissue to be sutured;
a fluid flow path;
a fluid inlet whereby a fluid under pressure can be fed into the fluid flow path;
an outlet whereby the fluid can be fed from the body, the fluid flow path connecting the fluid inlet and the outlet in flow communication, and the outlet opening out of the body at a position proximate the tissue penetration means;
a suture element feed path which opens into the fluid flow path via a suture element inlet, by which a suture element can be fed into the fluid flow path for displacement along the fluid flow path together with the fluid; and
releasable securing means for selectively releasing and securing a suture element received in the suture element feed path, for feed into the fluid flow path, thereby to permit control of the amount of suture element fed into the fluid flow path.

28. The implement as claimed in claim 27, in which the fluid inlet and the suture element inlet are provided by a single inlet into the fluid flow path.

29. The implement as claimed in claim 28, in which the body defines a reservoir, for holding the fluid to be fed into the fluid flow path, the reservoir being connected or connectable in flow communication with the fluid inlet.

30. The implement as claimed in claim 27, in which the suture element inlet opens into the fluid flow path at a position intermediate the fluid inlet and the outlet.

31. The implement as claimed in claim 27, which includes tissue drive means for driving the tissue onto the tissue penetrating means.

32. The implement as claimed in claim 27, in which the releasable securing means includes a securing rod which is displaceable within a bore, defined in the body at right angles to the suture element feed path and intersecting the suture element feed path, selectively to intercept or be removed from the suture element feed path.

33. The implement as claimed in claim 32, in which the securing rod is resiliently biassed towards a rest position, in which it intercepts the suture element feed path, by resilient bias means.

34. The implement as claimed in claim 33, in which the resilient bias means is provided by a diaphragm over a bore opening which leads into the bore, the diaphragm being displaceable outwardly under pressure of a fluid thereby to displace the securing rod and withdraw it, at least partly, from the suture element feed path to release the suture element for feed along the suture element feed path.
